(19) United States
(12) Patent Application Publication
(10) Pub. No.: US 2003/0070204 A1
(43) Pub. Date: Apr. 17, 2003

(54) Protective gear for crawlers and toddlers

(76) Inventor: Chauncey L. Summers, Tulsa, OK (US)

Correspondence Address: Chauncey L. Summers, 535 E. Latimer Ct., Tulsa, OK, 74106, US

(21) Appl. No.: 09/977,630

(22) Filed: Oct. 12, 2001

(51) Int. Cl. A61F 9/00; A41D 13/08

(52) U.S. Cl. 2/16

(57) ABSTRACT

Soft spandex™ (el DU Pont de Nemours and Co.) Fittings 8, 12, and 18 with protective fixtures 10, 13, 14, 15, 16, 17, and 18 Attached by zig-zag stitching 11 in a manner wherein when worn the protective fixtures would cover and protect their designated body area due to stable attachment facilitated by the said underpinning spandex™ (el DU Pont de Nemours and Co.) components 8, 12, and 18.

### BACKGROUND

1. Field of Invention

This invention relates to medical, athletic, and recreational gear that protects from injury.

2. Description of Prior Art

Numerous medical, athletic, and recreation orientated companies produce wraps, gloves, kneepads, and helmets that protect humans from injury. These companies produce gloves to protect the skin and joints of the hand and also to provide grip for athletic endeavors, kneepads to protect the skin and joints of the knee, and helmets to provide protection of the head. These devices provide protection by the absorption of shock and insulation from friction due to contact with an object or surface while the object or surface or the subject wearing the devices are in motion. However, these devices are not scaled to the dimensions of crawling and toddling children nor tailored to their special needs. In addition, these devices are unaesthetic to the point that they do not make a fashion statement, in specific, they would not be worn solely for fashion. Consequently, protective gear for crawlers and toddlers facilitates protection for crawling and toddling children with an aesthetic element that makes it fashionable. The devices known heretofore in respect to crawling and toddling children suffer from a number of disadvantages:

(a) These devices are oversized and even if scaled down would be bulky, cumbersome, and uncomfortable for crawling and toddling children due to their design and materials of construction. Protective gear for crawlers and toddlers is constructed from dyeable spandex™ (el DU Pont de Nemours and Co.) and goretex™ (W. L. gore Inc.) with durable stitching facilitating washability, maximum comfort and flexibility combined with optimum protection and a superior level of aesthetic value.

(b) The devices known heretoforth are not aesthetic or comfortable to the point where they would be worn solely for fashion. Protective gear for crawlers and toddlers, due to it's design and construction of dyeable spandex™ (el DU Pont de Nemours and Co.) and goretex™ (W. L. gore Inc.) is fashionable and trendy gear that can be decorated with a myriad of themes, parents would be pleased to have their children wear protective gear for crawlers and toddlers even in situations where their need for protection is minimal.

### SUMMARY

In context with presently known devices in this field, protective gear for crawlers and toddlers due to its' design and materials of construction, facilitates protection for crawling and toddling children, is easily laundered, is especially tailored to their needs, and has a superior degree of comfort and aesthetic quality.

### OBJECTS AND ADVANTAGES

A brief delineation of objects and advantages of protective gear for crawlers and toddlers described above:

(a) To provide protection for crawling and toddling children from their most common injuries.

(b) To facilitate an aesthetic quality giving value as fashion as well as providing protection.

(c) Protective gear for crawlers and toddlers is lightweight, and resilient due to its' design and the materials of its' construction.

(d) Protective gear for crawlers and toddlers is comfortable and unbinding due to its' design and the materials of its' construction therefore the child will not have a tendency to remove the gear at the behest of discomfort.

(e) Protective gear for crawlers and toddlers is constructed from materials that are impervious to water which also can be additionally treated with waterproofing chemicals therefore is ideal for facilitating protection while swimming or wading. Further objectives and advantages of protective gear for crawlers and toddlers is that it has longevity due to construction utilizing resilient materials and durable stitching making it viable as a “hand me down” and as the materials of its' construction are dyeable, protective gear for crawlers and toddlers can be decorated in accord with a variety of themes.(sports teams, military branches, popular organizations etc.) In addition, protective gear for crawlers and toddlers is made from materials that are washable and elastic therefore is easily laundered and has one-size-fits-all capability.

(f) Protective gear for crawlers and toddlers innovative protective spandex™ (el DU Pont de Nemours and Co.) Ear protector prevents foreign objects from entering the childs' ear without any impedance to hearing.

### DRAWING FIGURES

In the drawings closely related figures have the same number but different alphabetic suffixes.

FIGS. 1-1c shows various aspects of the protective gear for crawlers and toddlers knee protector, a soft spandex™ (el DU Pont de Nemours and Co.) strip symmetrically tapered on each end with concave incisions on each tapered end which are sown together to form a band with an oval space in front that is fitted with an oval goretex™ (W. L. gore Inc.) pad slightly larger than, and aligned with, the oval space in the band which is sown to the band with zig-zag stitching in a manner to cover the oval space in the band.

FIG. 2 shows soft spandex™ (el DU Pont de Nemours and Co.) strip with concave incisions on each tapered end.

FIG. 3 shows oval goretex™ (W. L. gore Inc.) pad.

FIG. 4-4b shows the various aspects of the protective gear for crawlers and toddlers' head protector, a band constructed of a soft spandex™ (el DU Pont de Nemours and Co.) strip with tabs extending laterally from the central third of the strip about the distance of half the width of the strip, symmetrically folded along its' longitudinal axis with goretex™ (W. L. gore Inc.) sections to cover the outer surface, and spandex™ (el DU Pont de Nemours and Co.) ear protectors fitted in both sides of the band at the point where the sown together tabs of the strip tapers into the thinner part of the band.

FIG. 5 shows a (relatively) small square goretex™ (W. L gore Inc.) section.

FIG. 5a shows a larger tapered polyhedral goretex™ (W. L. gore Inc.) section.

FIG. 5b shows a larger square goretex™ (W. L. gore Inc.) section.

FIG. 6 shows a spandex™ (el DU Pont de Nemours and Co.) ear protector.

FIG. 7 shows a soft spandex™ (el DU Pont de Nemours and Co.) section forming the back of the protective gear for crawlers and toddlers' hand protector, hand shaped (with shortened fingers), with and oblong extension between the thumb and index finger which forms the palm of hand protector.

FIG. 7a shows a goretex™ (W. L. gore Inc.) section, hand shaped with (shortened fingers) and an empty palm space, which forms the inner/front of glove.

FIG. 7b shows the protective gear for crawlers and toddlers' hand protector, a hand shaped soft spandex™ (el DU Pont de Nemours and Co.) section with an oblong extension (FIG. 7) with a hand shaped goretex™ (W. L. gore Inc.) section with palm missing (FIG. 7a) sown to the inner/front of the spandex™ (el DU Pont de Nemours and Co.) section with the oblong extension of the spandex™ (el DU Pont de Nemours and Co.) part sown onto the goretex™ (W. L. gore Inc.) section (FIG. 7a) in a manner as to cover the empty palm space of the goretex™ (W. L. gore Inc.) section.

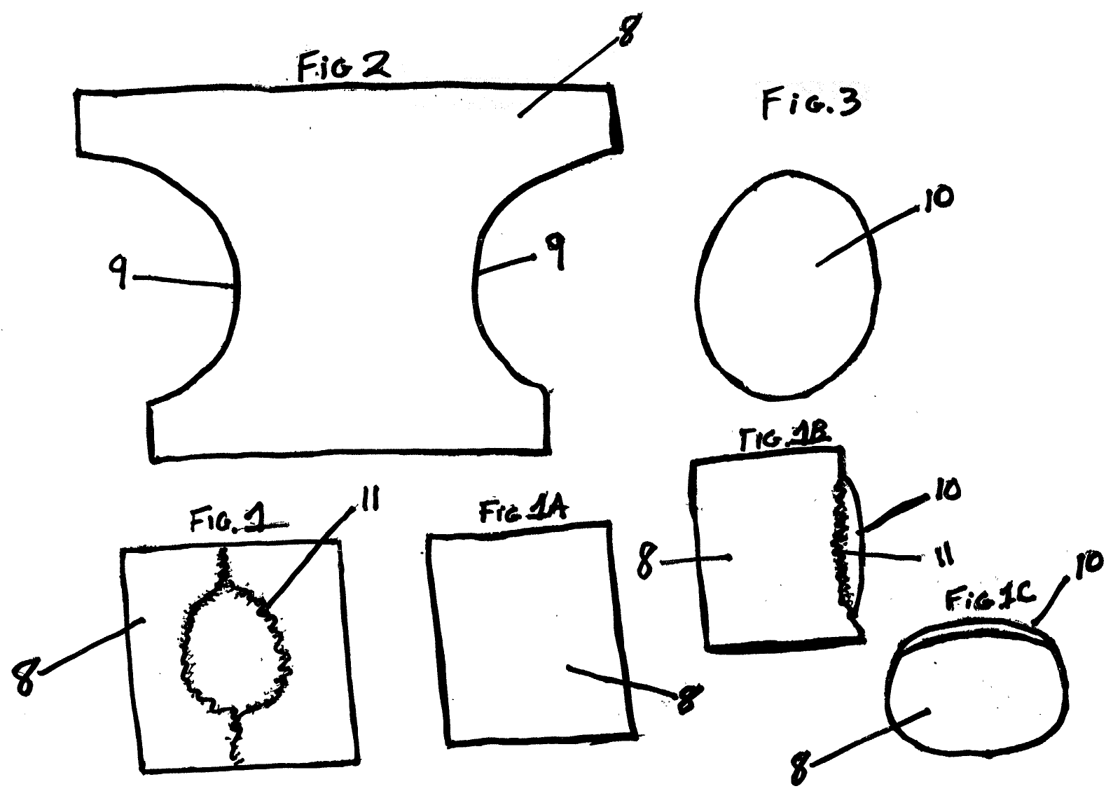

### REFERENCE NUMERALS IN DRAWINGS

8. Spandex™ (el DU Pont de Nemours and Co.) band

9. Concave incision

10. Goretex™ (W. L. gore Inc.) pad

11. Zig-zag stitching

12. Spandex™ (el DU Pont de Nemours and Co.) strip w/taps

13. Tabs

14. Small square goretex™ (W. L. gore Inc.) section

15. Larger tapered polyhedral goretex™ (W. L. gore Inc section

16. Larger square goretex™ (W. L. gore Inc.) section

17. Spandex™ (el DU Pont de Nemours and Co.) ear protector

18. Spandex™ (el DU Pont de Nemours and Co.) outer/back of hand protector w/oblong palm extension

19. Goretex™ (W. L. gore Inc.) inner/front of hand protector w/empty palm space

20. Oblong palm extension

21. Empty palm space

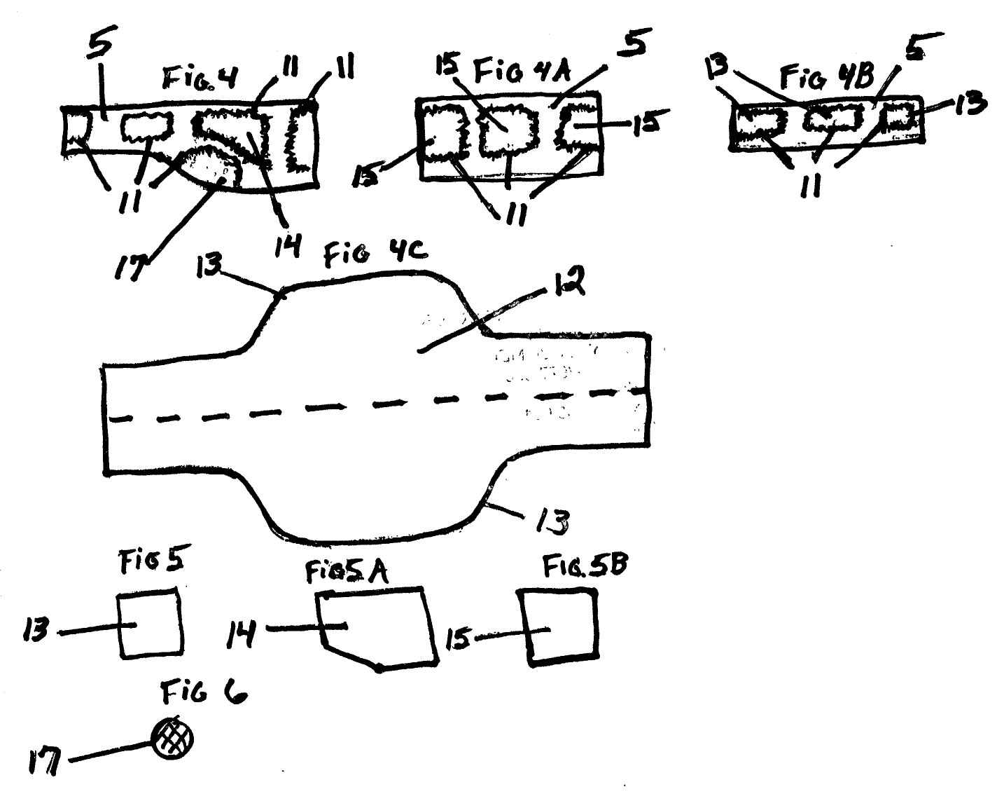

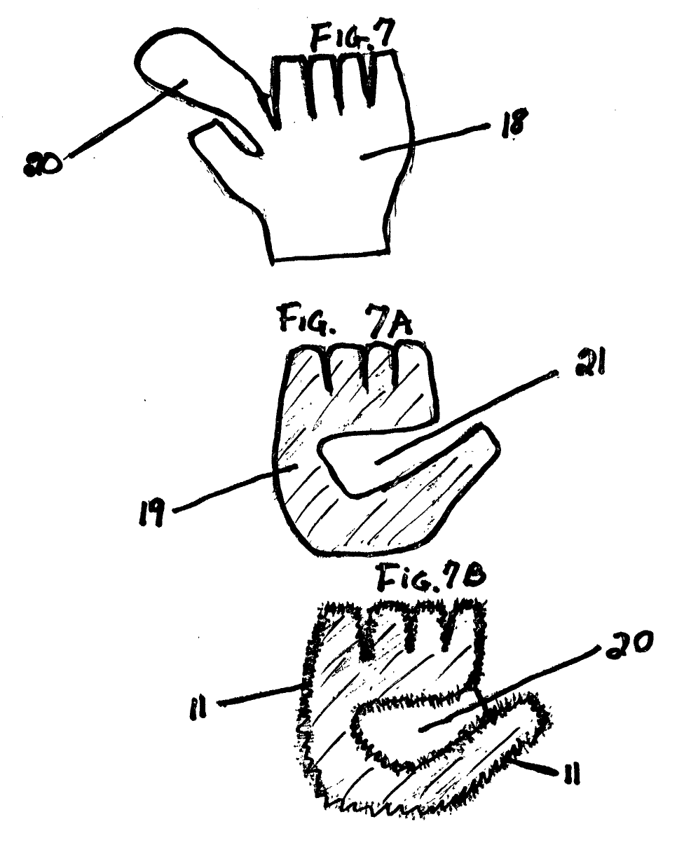

FIGS. 4-4b, 7-7b Additional Embodiments

Additional embodiments of the protective gear for crawlers and toddlers are shown in FIG. 4 (side view), FIG. 4a (rear view), and FIG. 4b front view). these figs show a strip of soft spandex™ (el DU Pont de Nemours and Co.) with tabs 13 symmetrically folded along its' longitudinal axis and sown together with zig-zag stitching 11 with small goretex™ (W. L. gore Inc.) sections 7 sown on with zig-zag stitching 11 in a manner as to cover the narrow part of the head protector that comprises the front with about ⅛″ space in between the sections. The tapered side part of the head protector, which gradually differentiates the narrow front part from the wider rear part formed from tabs 13 being sown together, has a larger polyhedral goretex™ (W. L. gore Inc.) section 15 sown onto the head protector covering the area of said tapers with the same ⅛″ spacing and proportion of coverage in context with the narrow front part of the band. The larger square goretex™ (W. L. gore Inc.) section is sown on the thicker rear part of the head protector with the same ⅛″ spacing and proportion of coverage in context with the tapered side part and the narrow front part. The spandex™ (el DU Pont de Nemours and Co.) ear protector 17 is sown onto both sides under the tapered side part of the head protector, with zig-zag stitching, 11 positioned to cover the child's ears. The remaining embodiment is illustrated in FIGS. 7-7b. FIG. 7a shows the hand protector, the spandex™ (el DU Pont de Nemours and Co.) outer/back of hand protector w/oblong palm extension, 18 with a hand shaped goretex™ (W. L. gore Inc.) section w/empty palm space, 19 sown into the inner part of the spandex™ (el DU Pont de Nemours and Co.) outer/back of hand protector w/oblong palm extension 18 and the oblong palm extension 20 sown onto the goretex™ (W. L. gore Inc.) inner/front of hand protector w/empty palm space 19 with zig-zag stitching 11 in a manner as to cover the empty palm space 21.

### ADVANTAGES

From the description above, a number of advantages of protective gear for crawlers and toddlers become evident:

(a) Construction from soft spandex™ (el DU Pont de Nemours and Co.) and goretex™ (W. L. gore Inc.) makes protective gear for crawlers and toddlers easily laundered.

(b) Construction from soft spandex™ (el DU Pont de Nemours and Co.) and goretex™ (W. L. gore Inc.) makes protective gear for crawlers and toddlers comfortable so children will not be inclined to remove it due to discomfort.

(c) Construction from elastic spandex™ (el DU Pont de Nemours and Co.) gives protective gear for crawlers and toddlers one-size-fits-all capability.

(d) The materials of construction are impervious to water and can be further waterproofed by chemical treating therefore protective gear for crawlers and toddlers is ideal for facilitating protection while swimming or wading.

(e) Protective gear for crawlers and toddlers prevents foreign objects from entering the ear without diminishing the childs' hearing.

### OPERATION

The manner of using protective gear for crawlers and toddlers is basicly the same as the use of gloves, kneepads, and helmets. The hand protectors are pulled over the fingers onto the hand until snug, with the goretex™ (W. L. gore Inc.) part on the inner/palm side of the hand. The knee protectors are pullet over the foot past the calf until positioned over the kneecap with the goretex™ (W. L. gore Inc.) part covering the kneecap. The head protector is slipped over the head, positioned horizontally level across the forehead, with the wider area positioned on the rear of the head and the narrower part positioned on the front of the head, and the ear protectors positioned over the ears. The elasticity of the spandex™ (el DU Pont de Nemours and Co.) holds the head protector onto the childs' head.

### CONCLUSION RAMIFICATIONS, AND SCOPE

This specification makes evident that protective gear for crawlers and toddlers will provide optimal protection from the most common injuries faced by crawlers and toddlers, is designed and constructed for their special needs, and in addition is comfortable, durable, impervious to water, fashionable, and has one-size-fits-all capacity. Protective gear for crawlers and toddlers has additional advantages in that:

it provides piece of mind for parents as well as protection for crawlers and toddlers. It is of simple construction and ea produced on any level, from being individually hand made to being mass produced in garment factories. It facilitates optimum grip for the smaller, weaker, and less coordinated hands of crawlers and toddlers.

Although the description above expresses many specifics, they should not be construed in a manner as to limit the scope of protective gear for crawlers and toddlers, but as merely exposing examples of some of the presently preferred embodiments thereof. For example: the padding can be made in varying shapes, decrees of thickness, or from different materials as long as they facilitate protection, comfort, aesthetic quality, and the other essential elements of protective gear for crawlers and toddlers. The protective gear for crawlers and toddlers can have ornamental attachments as long as they do not impede the safety objectives. the hand protectors can be made with full fingers as opposed to the exposed fingertip version. Protective gear for crawlers and toddlers can be modified to fit any part of the body; examples of this, the knee protectors can be used as elbow protectors or a totally different embodiment can be designed and constructed for other parts of the body. Protective gear for crawlers and toddlers can be specially constructed for children with special physical needs, and can be treated with waterproofing chemicals where applicable.

I claim:

1. Protective gear for crawlers and toddler is gear comprised of protective fixtures and elastic material that is dyeable and impervious to water. The elastic material provides means of attaching to and stabilizing the protective fixtures over any area of the human body. The improvement wherein protective gear for crawlers and toddlers is the design and materials of construction that facilitate protection with an unprecedented level of viability and comfort suitable for crawling and toddling children, novel fashionablity, and ease of laundering.
